(19) United States
(12) Patent Application Publication
(10) Pub. No.: US 2012/0135401 A1
(43) Pub. Date: May 31, 2012

(54) Methods and Compounds for the Diagnosis of Inflammatory Disease and Identification of Pharmacological Agents Useful in the Treatment of Inflammatory Disease

(75) Inventors: Jun LI, Danbury, CT (US); Xiang John LI, Danbury, CT (US); Randall W. BARTON, Farmington, CT (US)

(73) Assignee: BOEHRINGER INGELHEIM PHARMACEUTICALS, INC., Ridgefield, CT (US)

(21) Appl. No.: 13/069,739

(22) Filed: Mar. 23, 2011

Related U.S. Application Data
(63) Continuation of application No. 12/567,251, filed on Sep. 25, 2009, now abandoned.
Application No. 12/567,251 is a continuation of application No. 11/281,158, filed on Nov. 17, 2005, now abandoned.
Application No. 11/281,158 is a division of application No. 10/154,506, filed on May 23, 2002, now abandoned.
(60) Provisional application No. 60/333,848, filed on Nov. 28, 2001.
(60) Provisional application No. 60/335,474, filed on Nov. 15, 2001.
(60) Provisional application No. 60/292,968, filed on May 23, 2001.

(51) Int. Cl. C12Q 1/68

(52) U.S. Cl. 435/6.11

(57) ABSTRACT

Methods for the diagnosis of inflammatory bowel diseases and the identification of agents useful in the treatment of such diseases based upon the agent's effect on reducing Pim-2 expression.

### RELATED APPLICATIONS

This application is a continuation of U.S. Continuation application Ser. No. 12/567,251, filed on Sep. 25, 2009 which is a continuation of U.S. Divisional application Ser. No. 11/281,158 filed on Nov. 17, 2005, which claims priority of U.S. Non-Provisional application Ser. No. 10/154,506 filed May 23, 2002, which claims priority from U.S. Provisional application No. 60/292,968 filed May 23, 2001; U.S. Provisional application No. 60/335,474 filed Nov. 15, 2001; and U.S. Provisional application No. 60/333,848 filed Nov. 28, 2001.

### BACKGROUND OF THE INVENTION

1. Field of the Invention

The field of this invention relates to the area of molecular biology. In particular, the present invention relates to the polynucleotide sequence encoding human Pim-2 (h-Pim-2) and the corresponding translated h-Pim-2 polypeptide, recombinant vectors comprising h-Pim-2 nucleic acid sequence, and methods for recombinant production of h-Pim-2 polypeptides, as well as the use of the same in diagnosing inflammatory disease states and in screening assays for identification of compositions that may be useful in the treatment of inflammatory disease states, in particular inflammatory bowel diseases such as ulcerative colitis and Crohn's Disease.

2. The Related Art

Pim-2 is a highly conserved serine/threonine kinase involved in cell proliferation, meiosis and the prevention of apoptosis (Baytel et al., Biochim. Biophys. Acta Gene Struct. Expr. 1442: 274 (1998)). Pim-2 of mice, also known at Tic-1, has been reported to be about 53% identical in sequence at the amino acid level to the proto-oncogene Pim-1, and to be expressed at low levels in a variety of tissues, with the highest expression in the brain and thymus (van der Lugt et al., EMBO J. 14(11): 2536 (1995)). Like Pim-1, the Pim-2 locus is also a common site of provirus integration (Haupt et al., Cell 65: 753 (1991); Bruer et al., Embo J. 8: 743 (1989)). In fact, Pim-2 was first identified by means of proviral tagging experiments carried out in mice, and analysis of DNA obtained from outgrown tumors obtained after transplantation of primary lymphomas induced by inoculation of newborn BABL/c or C57BL10 mice in which the Pim-1 gene was largely deleted by gene targeting with Moloney MuLV. (Breuer et al., Embo J. 8(3): 743 (1989)). Such studies suggest that Pim-2 is a proviral integration site that carries somatically acquired proviruses in the majority of transplanted tumors (Id.).

The Pim-1 proto-oncogene is believed to be one of the most potent collaborators of myc proto-oncogenes in inducing lymphomagenesis in mice (van der Lugt et al., EMBO J. 14(11): 2536 (1995)). Allen et al. (Oncogene 15: 1133 (1997)) suggest, based on proviral tagging experiments, that Pim-2 is similar in oncogenic behavior to Pim-1. They note that while basal expression of Pim-1 and Pim-2 differ with respect to basal expression in tissues, that both genes are highly expressed in response to the same cytokines. A Pim-2 transgene in lymphoid cells was seen to predispose mice to T-cell lymphomas like those promoted by pim-1 transgenes.

As iterated above, Pim-2, as the related Pim-1 gene, encodes labile, cytoplasmic serine/threonine kinases. Phosphorylation of protein substrates by serine/threonine kinases is often involved in the transduction of signals from the cell surface receptors to intracellular effectors. It is believed that Pim-2, like Pim-1, is a target for gp130-mediated signal transducer and transcriptional activator 3 (“STAT3”) signaling. As is known to those of ordinary skill in the art, the activation of STAT3 by the cytokine receptor gp130 is required for both G1 to S cell cycle transition, as well as, anti-apoptosis (Shirogane et al., Immunity 11: 709 (1999)).

Baytel et al. (Biochim Biophys. Acta Gene Struct. Expr. 1442: 274 (1998)) report cloning of the h-Pim-2 gene. In comparison to mouse Pim-2, h-Pim-2 is reported by Baytel et al. to encode a protein that shares 90% identity and 93% similarity at the primary structure level. At the RNA level, two Pim-2 transcripts have been identified in humans, a 2.2 kb transcript that is highly expressed in hematopoietic tissues and in leukemic and lymphoma cell lines, and a 5.0 kb transcript that is detectable in spleen, thymus, small intestine and colon apoptosis (Baytel et al., Biochim. Biophys. Acta Gene Struct. Expr. 1442: 274 (1998)). The Pim-2 gene in humans is believed to be X-linked (van der Lugt et al., EMBO J. 14(11): 2536 (1995)).

The present inventors (Li et al., J. Biol. Chem. 276: 18579 (2001)) have recently disclosed that Pim-2 is induced by lipopolysaccharide (LPS) in a variety of cell lines. Studies undertaken by the inventors suggest that up-regulation of Pim-2 in 70Z3 cells by LPS is controlled by the IKK/NF-κB pathway.

Aberrant protein serine/threonine activity has been implicated, or is suspected in a number of pathologies including septic shock, bone loss, psoriasis, rheumatoid arthritis, many cancers and other proliferative diseases (See, U.S. Pat. No. 6,165,716 to Creasy et al. (Issue Date: Dec. 20, 2000)). A number of researchers have expended considerable time to identify serine/threonine protein kinases that may play a role in preventing, ameliorating and correcting dysfunctions or diseases. For example, U.S. Pat. No. 5,972,606 to Creasy et al. (Issue Date: Oct. 26, 1999), discloses a human protein serine/threonine kinase, designated HOACF72, of the hYAK1 family of polypeptides, antibodies against which are said to be useful in the treatment of bone loss, inflammatory diseases such as rheumatoid arthritis, osteoarthritis, adult respiratory disease syndrome (ARDS), inflammatory bowel disease (IBD), psoriasis, dermatitis, asthma, allergies, infections, septic shock, pain, cancers, anorexia, bulimia, and a host of other conditions. U.S. Pat. Nos. 5,965,420 and 6,165,766, also to Creasy et al. (Issue Dates: Oct. 12, 1999 and Dec. 26, 2000, respectively), assert human YAK3 polypeptides and polynucleotides, antibodies against which are said to be useful for treating bone loss, inflammatory diseases, infections, immunodeficiency disorders, septic shock, pain, cancers and a host of other pathological conditions. As stated by Creasy et al., there is a need for further identification and characterization of further members of the serine/threonine protein kinase family to identify other members of the family that may play a role in preventing, ameliorating or correcting dysfunctions or diseases. There is also a need to identify potential relationships between these kinases and disease states themselves.

### SUMMARY OF THE INVENTION

The prior art has emphasized the role of Pim-2 in oncogenic behavior, and has classified the gene as a proto-oncogene. It was particularly surprising that the present inventors have found that transcription of Pim-2 is significantly increased in a variety of inflammatory states, with particularly large increases in Pim-2 mRNA seen with respect to intestinal tissue levels in patients diagnosed with ulcerative colitis and Crohn's disease and inflammatory disease states associated with an inflamed: pancreas, tonsils, bowel (including small and large intestines and rectum), stomach lining, thyroid, cervix, lung, kidney, liver, and skin.

The present invention relates to polynucleotide sequences encoding human Pim-2 (h-Pim-2) and h-Pim-2 polypeptides. One embodiment of the invention relates to methods for using such polynucleotides and polypeptides for the treatment of human inflammatory diseases, such as ulcerative colitis and Crohn's Disease. Another embodiment of the invention relates to methods for screening compounds for potential anti-inflammatory activity by adjudging the effect of such compounds on Pim-2 activity. And yet another embodiment of the present invention relates to diagnostic assays for detecting inflammatory diseases associated with altered Pim-2 activity.

In one embodiment of the present invention, there is disclosed a method for diagnosing inflammatory disease states, such as an inflammatory bowel disease, using a tissue sample obtained from a patient, said method comprising the steps of: (a) measuring the level of Pim-2, or Pim-2 mRNA, in the tissue sample of the patient; and (b) determining any difference of the level of Pim-2 or Pim-2 mRNA in the tissue sample of the patient as compared to the level of Pim-2 or Pim-2 mRNA in comparable tissue sample(s) obtained from one or more patients lacking the inflammatory disease state. Such method may further comprise the step of (c) diagnosing the patient as having the inflammatory disease state when the measurement of such parameter with respect to the patient's tissue is significantly higher than in comparable tissue sample(s) obtained from the one or more patients lacking the inflammatory disease state. By “significantly higher” it is meant a difference of more than about 50%, more preferably more than about 100%, and yet more preferably more than about 200%. The level of Pim-2 or Pim-2 mRNA may be measured directly, or indirectly, as for example by measuring kinase activity of Pim-2. Such method may comprise in situ hybridization of at least one nucleic acid probe comprising a polynucleotide sequence of at least about 15 contiguous nucleotides of SEQ ID NO:1, preferably a nucleic acid probe includes nucleotides 294 through 311 of SEQ ID NO:1. Such method may be particularly advantageously used to diagnosis Crohn's Disease and ulcerative colitis, but may also be used to detect inflammatory disease states associated with an inflamed pancreas, tonsils, bowel (including small and large intestines and rectum), stomach lining, thyroid, cervix, lung, kidney, liver, and skin.

In another embodiment of the present invention, there is provided a method for diagnosing an inflammatory disease state, such as an inflammatory bowel disease, in a patient comprising the steps of: (a) establishing a statistically significant correlation between Pim-2 expression in the inflamed tissue of the inflammatory disease state, and the presence and/or severity of the inflammatory disease state; (b) measuring the Pim-2 level in corresponding tissue obtained from said patient; and (c) determining whether the measured Pim-2 level corresponds to a level correlated with the inflammatory disease state. Such method may also be particularly advantageously used to diagnosis Crohn's Disease and ulcerative colitis, but may also be used to detect inflammatory disease states associated with an inflamed pancreas, tonsils, bowel (including small and large intestines and rectum), stomach lining, thyroid, cervix, lung, kidney, liver, and skin.

There is also provided a method for monitoring the efficacy of anti-inflammatory drug regimens in the treatment of an inflammatory disease state, such as an inflammatory bowel disease, said method comprising the steps of: (a) establishing a statistically significant correlation between Pim-2 levels and clinical response to anti-inflammatory therapy in the inflammatory disease state; (b) measuring the Pim-2 level in the patient; and (c) determining the correspondence between the Pim-2 level measured in the patient and the Pim-2 levels correlated to clinical response to anti-inflammatory therapy. This method may advantageously be employed to monitor the efficacy of anti-inflammatory drug regimens with respect to the treatment Crohn's Disease and ulcerative colitis. This method may also be employed to monitor the efficacy of anti-inflammatory drug regimens with respect to inflammatory disease states associated with an inflamed pancreas, tonsils, bowel (including small and large intestines and rectum), stomach lining, thyroid, cervix, lung, kidney, liver, and skin.

Other methods for detecting inflammatory disease states, such as an inflammatory bowel disease, are also encompassed by the present invention. For example, the present invention further provides a method for detecting inflammatory disease states comprising the steps of: (a) collecting a suspect sample, and (b) subjecting the suspect sample to a diagnostic test employing the nucleotide sequence of SEQ ID NO:1, or fragments thereof, the diagnostic test comprising polymerase chain reaction or nucleic acid hybridization or (a) collecting a suspect sample, and (b) subjecting the sample to a diagnostic test comprising polyclonal antisera and/or monoclonal antibody raised to immunogens comprising the polypeptide sequence of SEQ ID NO:2, or immunogenic fragment thereof, said diagnostic test comprising Western blot analysis or enzyme-linked immunoassay (ELISA). It also provides a method for detecting inflammatory disease states comprising the steps of: (a) collecting a suspect sample, and subjecting the sample to a diagnostic test comprising polyclonal antisera and/or monoclonal antibody raised to immunogens comprising the polypeptide sequence of SEQ ID NO:2, or immunogenic fragment thereof, (b) detecting said polyclonal antisera and/or monoclonal antibody.

A diagnostic kit for detecting inflammatory disease states is also encompassed by the present invention. Such diagnostic kit may comprise, for example: (a) an antibody specific for SEQ ID NO:2 or an antigen-binding portion of an antibody specific for SEQ ID NO:2; and (b) reactants for detecting said antibody or portion specific for SEQ ID NO:2.

The present invention also provides for screening assays for determining whether a compound would be effective in the treatment of an inflammatory disease state. One such screening assay comprises the steps of: (a) incubating the compound with cells that express SEQ ID NO:2, or variant thereof, upon exposure to LPS; (b) determining the extent of inhibition caused by said compound on the expression of SEQ ID NO:2, or variant thereof, by measuring a parameter indicative of the level of SEQ ID NO:2 (or variant thereof) or m-RNA translated to SEQ ID NO:2 (or variant thereof). Another such screening assay comprises: (a) incubating in vitro the compound with a protein comprising SEQ ID NO:2, or variant thereof, having kinase activity, and a substrate with respect to said kinase activity; (b) determining whether the compound inhibits the kinase activity of the protein with respect to the substrate. The protein of this assay may be of recombinant or natural origin. Compounds identified by such screening assays are also encompassed by the present invention.

Another screening assay for identifying compounds that ameliorate inflammatory disease states comprises the steps of: (a) separately cultivating a first immortalized cell line containing at least one gene of SEQ ID NO:1, and a second immortalized cell line wherein the gene of SEQ ID NO:1 is inactivated; (b) subjecting both cell lines to a compound suspected of having anti-inflammatory activity; and (c) determining if said compound selectively inhibits growth of said first immortalized cell line. Again, compounds identified by such assay are within the scope of the present invention.

And yet in another embodiment of the present invention, there is provided a screening assay (and compounds identified thereby) for identifying compounds that may have use in the amelioration of inflammatory disease states, such as an inflammatory bowel disease, due to modulation or alteration of Pim-2 activity, comprising the steps of: (a) establishing a control system comprising Pim-2 and a substrate of Pim-2; (b) establishing a test system comprising Pim-2, said substrate of Pim-2 and the candidate compound; (c) measuring the activity of Pim-2 in the control and test systems; and (d) determining that the candidate compound modulates or alters Pim-2 activity if the activity of Pim-2 in the test system is less than or greater than the activity measured for the control system. The screening assay may also comprise contacting a compound with a cultured cell that expresses the Pim-2 gene, and detecting a change in the expression of the Pim-2, or kinase activity of Pim-2, in the cultured cell. This method may further comprise the step of—determining that a screened compound is useful in the treatment of inflammatory disease states when the expression of the Pim-2 gene, or kinase activity of Pim-2, in the cultured cell is significantly diminished by the screened compound. By “significantly diminished” it is meant that the expression of the Pim-2 gene, or kinase activity of Pim-2, is reduced by more than about 50%, more preferably 100%, and yet more preferably 200%. These methods may also be employed for identifying compounds that may have use in the amelioration of inflammatory disease states associated with an inflamed pancreas, tonsils, bowel (including small and large intestines and rectum), stomach lining, thyroid, cervix, lung, kidney, liver, and skin.

Alternatively, compounds can be screened for activity in the treatment of inflammatory disease states and inflammatory disease states associated with an inflamed pancreas, tonsils, bowel (including small and large intestines and rectum), stomach lining, thyroid, cervix, lung, kidney, liver, and skin by measuring the affinity of the compounds for Pim-2.

In the screening methods of the present invention, a change in the expression level of pro-inflammatory cytokines, such as IL-6, compared to control is an indication of Pim-2 activity. Differences in expression levels may be determined using methods known in the art including but not limited to RNA interference (RNAi) technology (Elbashir, S. M. et al, 2001, Nature, 411, 494-498).

Candidate compounds identified and/or isolated by any of these methods are also encompassed by the present invention.

Methods for treating animals inflicted with inflammatory disease states, and preventing the development of inflammatory disease states, including but not limited to an inflammatory bowel disease, are also disclosed.

In one method, treatment is accomplished by administration of a therapeutically or prophylactically effective amount of an antisense compound targeted to a nucleic acid sequence encoding Pim-2. In yet another embodiment, there is provided a method for treating an inflammatory disease state, such as an inflammatory bowel disease, which comprises administering to a patient in need thereof an oligonucleotide which specifically hybridizes to a transcript encoding human Pim-2 and suppresses the expression of the human Pim-2, as its effective ingredient, and a pharmacologically acceptable carrier. In another method, there is administered to a patient an amount of an agent that inhibits Pim-2 production, wherein the agent is an antisense construct that targets Pim-2 encoding sequences, under conditions that the treatment is effected. In another method, the agent is a short interfering (si) RNA construct that targets Pim-2 encoding sequences.

The above methods of diagnosing, monitoring efficacy of antiinflammatory drugs, detecting, screening, and identifying work particularly well with respect to inflammatory bowel diseases, in particular Crohn's Disease and Ulcerative Colitis.

### BRIEF DESCRIPTION OF THE DRAWINGS

FIG. 1 shows the fold changes of Pim-2 mRNA expression in persons suffering different inflammatory disease states as compared to Pim-2 mRNA expression in persons lacking such inflammatory disease states. The number of donor samples tested is indicated by (n). mRNA expression levels were obtained by Affymetrix Gene Chip arrays as described (Lockhart, D. J. et al., Nat. Biotechnol. 14: 1675-1680 (1996)). Confidence p-values were calculated based on a two-sided Welch modified two-sample t-test.

FIG. 2 shows Pim-2 m-RNA expression in inflamed bowel tissue as compared to Pim-2 mRNA expression in non-inflamed bowel tissue of the same patients diagnosed with ulcerative colitis. “A” indicates “inflamed” and “B” indicates “non-inflamed” tissue. The four different patients are numbered as 1, 2, 3, and 4.

FIG. 3 shows the results of three experiments wherein Pim-2 expression was measured in THP-1 cell lines stimulated and unstimulated with lipopolysaccharide. Fold change values were derived from the comparison of Pim-2 mRNA expression in THP-1 cells stimulated with LPS for 6 hours versus Pim-2 mRNA expression in unstimulated THP-1 cells.

FIG. 4 shows Pim-2 mRNA is induced by anti-CD3 or IL-12/IL-18 stimulation in CD4+ Th1 cells. D011.10 splenic cells were stimulated with OVA, IL-12 and anti-IL-4 for 7 days. CD4+ cells were harvested and stimulated with anti-CD3 or IL-12/IL-18 for 16 hours; the total RNA was extracted and first strand cDNA was synthesized. The mRNA of Pim-2 and IFN-γ were detected by TaqMan analysis. The mRNA expression levels of each gene are presented as percentage values of the mean mRNA copy number in IL-12/IL-18 stimulated sample.

FIG. 5 shows that purified recombinant Pim-2 is active in phosphorylating Histone. His-tagged Pim-2 was expressed and purified from E. coli. Indicated amounts of Pim-2 were assayed in a buffer containing 25 mM HEPES pH 7.5, 10 mM MgCl₂, 0.5 mM DTT, 10 μM cold ATP, 1.5 μCi [γ-³³P]-ATP, 10 μg Histone III (type ss from calf thymus) at room temperature (--μg enzyme vs. His-Pim-2, 15 min.; ••••μg enzyme vs. His-Pim-2, 30 min.; -▾-μg enzyme vs. His-Pim-2, 60 min.). Incorporation of ³³P into Histone was measured.

FIG. 6 shows that Pim-2 is required for TNF-α-induced IL-6 expression in HeLa cells. Pim-2 siRNA duplexes (PIM2_{—}1 inverted control and PIM2_{—}1 at 200 nM) were transfected into HeLa cells for 2 days. Then, the cells were treated with various concentrations (between 0.16 and 20 ng/ml) of TNF-α for 2 hours before their total cellular RNA was prepared for TaqMan real-time PCR analysis. RT and PCR were carried out using TaqMan quantitation (showing mRNA copy numbers detected in 20 ng total RNA). The copy numbers of gene transcripts were determined according to DNA standards and normalized with human Gapdh. All TaqMan PCR reactions of each individual sample were performed in triplicate, then the copy numbers and standard error were determined.

### DETAILED DESCRIPTION OF THE INVENTION

### 1. Definitions

The following definitions are provided to facilitate understanding of certain terms used herein:

By “antibodies” it is meant to include polyclonal and monoclonal antibodies, chimeric, single chain, and humanized antibodies, as well as Fab fragments, including the product of an Fab or other immunoglobulin expression library.

By “cells” it is meant to include cells in any form, including, but not limited to, cells retained in tissue, cell clusters and individually isolated cells.

By “cell line” it is meant a clone of a primary cell that is capable of stable growth in vitro for many generations.

By “clone” it is meant a population of cells derived from a single cell or common ancestor by mitosis.

By a DNA “coding sequence” it is meant a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus A polyadenylation signal and transcription termination sequence will usually be located 3′ (downstream) to the coding sequence.

By “exogenous” material it is meant material that has been introduced into a cell, organism etc. that originated outside of the same.

By “heterologous” region of a DNA construct it is meant an identifiable segment of DNA within a larger DNA molecule that is not found in association with the larger molecule in nature.

By “isolate” a material it is meant changing the environment of the material or removing a material from its original environment, or both. For example, when a polynucleotide or polypeptide is separated from the coexisting materials of its natural state, it is “isolated.”

By “operably linked” nucleotide sequences it is meant a juxtaposition such that the functionality of the sequences is preserved. Thus, for example, a coding sequence “operably linked” to a promoter is positioned so that the promoter is capable of effecting the expression of the coding sequence.

By “polynucleotide” it is meant any polyribonucleotide or polydeoxyribonucleotide, which may be unmodified RNA or DNA, or modified DNA or DNA. As used herein, “polynucleotide” include, without limitation, single- and double-stranded DNA and RNA, hybrid molecules comprising DNA and RNA that may be single-stranded, or more typically double-stranded, or a mixture of single- and double-stranded regions. The term “polynucleotide” further may refer to triple-stranded regions comprising RNA or DNA or both DNA and RNA. “Polynucleotide” embraces chemically, enzymatically or metabolically modified forms of polynucleotides typically found in nature, as well as chemical forms of DNA and RNA characteristic of viruses and cells. The term is meant to encompass both long nucleotide as well as short nucleotide sequences, often referred to as oligonucleotides, and oligomers.

By the term “polypeptide” it meant to refer to any peptide or protein comprising two or more amino acids joined to each other by peptide bonds or modified peptide bonds, i.e. peptide isosteres. Polypeptides may comprise amino acids other than the 20 gene-encoded amino acids, and includes amino acids modified either naturally or synthetically.

By “Pim-2 polypeptide” it is meant to include SEQ ID NO:2, and polypeptides comprising an amino acid sequence of SEQ ID NO:2 that have at least 80% identity, still more preferably 90% identity, and still more preferably 95% identity, with the sequence of SEQ ID NO:2 over its entire length. The Pim-2 polypeptide may be in the form of the “mature” protein or may be a part of a larger protein such as a fusion protein, and may include secretory or leader sequences, pro-sequences, sequences which aid in purification, or additional sequence for stability during recombinant production.

By “recombinant” or “engineered” cell it is meant a cell into which a recombinant gene has been introduced through the hand of man. Recombinantly introduced genes may be in the form of a cDNA gene (i.e., lacking introns), a copy of a genomic gene (i.e., including introns with the exons), genes produced by synthetic means, and/or may include genes positioned adjacent to a promoter, or operably linked thereto, not naturally associated with the particular introduced gene.

By “replicon” it is meant any genetic element (e.g., plasmid, chromosome, virus) that functions as an autonomous unit of DNA replication in vivo, i.e. capable of replication under its own control.

By “transformed cell” it is meant a cell into which exogenous or heterologous DNA has been introduced. The transforming DNA may or may not be integrated (covalently linked) into chromosomal DNA making up the genome of the cell. The transforming DNA may be maintained on an episomal element such as a plasmid.

By “variant” it is meant a sequence, such as a polynucleotide or polypeptide, that differs from another sequence, but retains essential properties. For example, a variant of a polynucleotide may differ in nucleotide sequence by one or more substitutions, additions, and deletions, from the reference polynucleotide.

By “vector” it is meant a replicon, such as a plasmid, phage or cosmid, to which another DNA segment may be attached so as to bring about the replication of the attached segment.

### 2. Diagnostic Assays for the Determination of Inflammatory Disease States

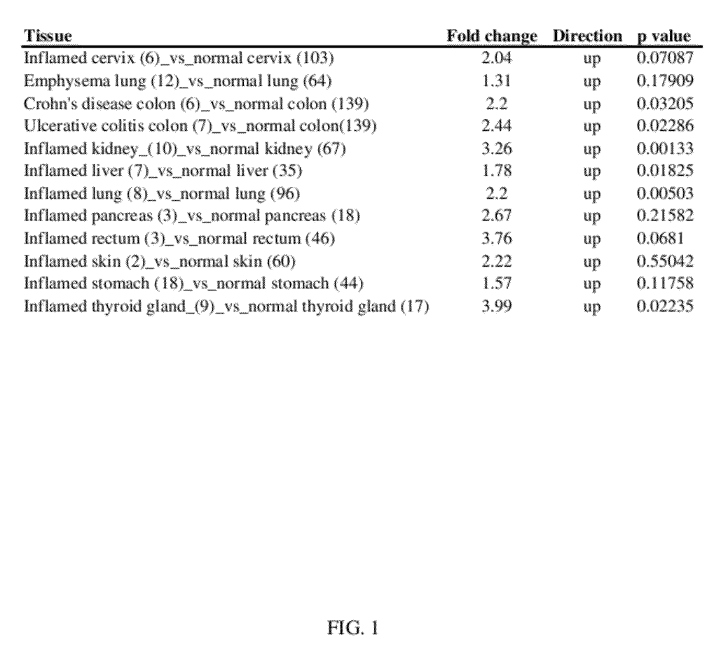

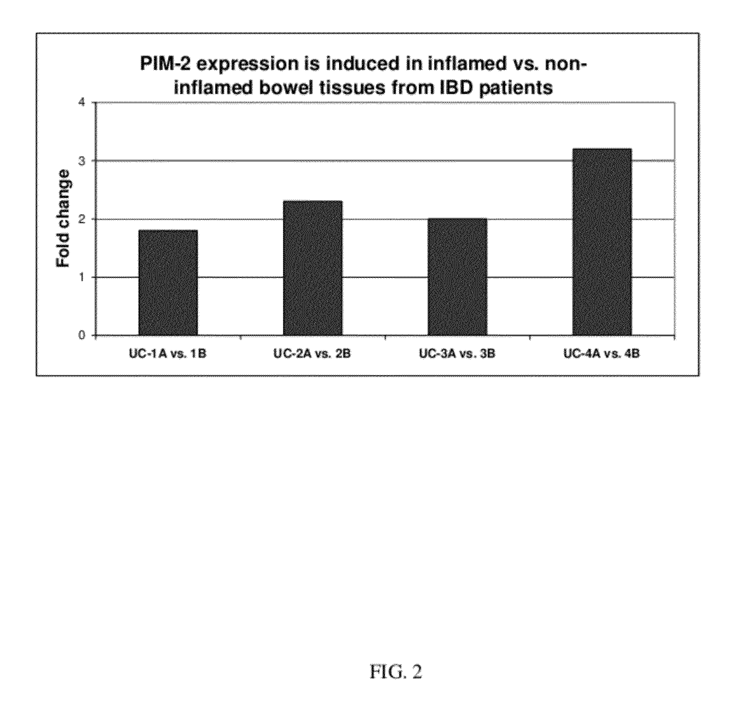

The present inventors have surprisingly discovered that Pim-2 transcription and translation is significantly enhanced in inflammatory disease states. In particular, as shown in FIG. 1, the present inventors have discovered that expression of h-Pim-2, and its m-RNA template, are dramatically increased in select tissues of humans diagnosed with ulcerative colitis and Crohn's disease, two inflammatory bowel diseases, inflamed thyroid diseases, inflamed stomach diseases, inflamed pancrease diseases, inflamed cervix, inflamed lung tissue, inflamed kidney, inflamed liver, and inflamed skin, as compared to tissue of humans without such inflammatory disease states or in remission from such inflammatory disease states (controls). Increases in expression of h-Pim-2, and its m-RNA template, have also been noted in tonsillitis, thyroiditis and inflamed rectal disease. As seen in FIG. 2, mRNA expression of h-Pim-2 was significantly higher (2-3 fold) in inflamed colon tissues of persons suffering from the inflamed bowel disease ulcerative colitis as compared to non-inflamed colon tissue.

Inflammatory diseases may be diagnosed by methods comprising determining from a sample derived from a subject the extent of transcription and translation of Pim-2 as compared to transcription and translation of the gene in a normal population (i.e., not suffering from the inflammatory disease). Characterization of expression at the RNA level may be made using any of the methods well known in the art for the quantization of polynucleotides, such as, for example, PCR, RT-PCR, RNase protection, Northern blotting and other hybridization methods. Levels of Pim-2 polypeptide can be assayed likewise using techniques well known to one of ordinary skill in the art, such techniques including, but not limited to, competitive-binding assays, Western Blot analysis and ELISA assays. Microarray technology is well known and has general applicability to gauge gene expression.

### 3. Screening Assays

The unexpected discovery that Pim-2 expression is significantly enhanced at both the transcriptional and translational level in inflammatory disease states proffers new screening procedures to isolate compounds that diminish (or increase) the severity of the inflammatory disease state.

Agonists or antagonists of Pim-2, or of the transcription and/or translation of the Pim-2 gene, may find use in the treatment of inflammatory states.

Antagonists may be employed for therapeutic and prophylactic purposes to decrease inflammation by decreasing Pim-2 activity in the affected tissue or organ. Antagonists of Pim-2 activity may find particular use in ameliorating inflammatory bowel diseases, inflammatory thyroid diseases, inflammatory stomach diseases, and inflammatory pancreas diseases wherein increased expression of the gene is seen to be high in affected individuals. Antagonists may also find use in ameliorating inflammatory conditions seen in other tissues, including, but not limited to, the lung, skin, kidney, and thyroid. As Pim-2 is expressed widely in tissues of the body, antagonists of Pim-2 baseline activity may find use in a variety of inflammatory states other than these inflammatory diseases, including (but not limited to) adult respiratory disease syndrome (ARDS), allergies, asthma, dermatitis, osteoarthritis, psoriasis, rheumatoid arthritis.

Screening procedures may entail utilization of appropriate cells that express Pim-2 or respond to Pim-2 polypeptide of the present invention. Such cells include cells from mammals, yeast, Drosophila or E. coli. One particularly useful cell-line is THP-1, a human acute monocytic leukemia cell line available from American Type Culture Collection, Rockville, Md. (USA) which displays lymphoblastic-like cell morphology, has Fc and C3b receptors and lack surface and cytoplasmic immunoglobulins (these cells stain positive for alfa-naphthyl butyrate esterase, produce lysozymes and are phagocytic). Cells that express Pim-2, or respond to Pim-2, may be contacted with a test compound to determine the effect of the test compound on Pim-2 activity. Test compounds demonstrating action to reduce Pim-2 activity with respect to such cells may be considered good candidates as therapeutic agents in treating inflammatory disease states.

Pim-2 expressing cells may be cells transformed so as to express SEQ ID NO:2, or a variant thereof. The Pim-2 polypeptide of SEQ ID NO:2 may be prepared in both prokaryotic and eukaryotic systems. Constructs may be made wherein the coding sequence for the polypeptide is preceded by an operable signal peptide which results in secretion of the protein. The particulars for construction of expression systems and purification of peptides, and cleavage from fusion peptides are well known to those of ordinary skill in the art. Technology for introduction of DNA into cells includes four general methods: (1) physical methods such as microinjection, electroporation and the gene gun (See, eg., Johnston et al., Gene gun transfection of animal cells and genetic immunization, 43(A) Methods Cell. Biol. 353-365 (1994)); (2) viral vectors (See, e.g., Eglitis et al., Retroviral vectors for introduction of genes into mammalian cells, 6(7) Biotechniques 608-614 (1988)); (3) chemical methods (See, e.g., Zatloukal et al., Transferrinfection: A highly efficient way to express gene constructs in eukaryotic cells, 660 Ann. N.Y. Acad. Sci. 136-153 (1992)), and (4) receptor-mediated mechanisms (See, e.g., Wagner et al., Coupling of adenovirus to transferrin-polylysine/DNA complexes greatly enhances receptor mediated gene delivery and expression of transfected genes, 89(13) Proc. Natl. Acad. Sci. USA 6099-6103 (1992)).

As would be understood by one of ordinary skill in the art, minor modification of the primary amino acid sequence of SEQ ID NO:2 may result in a polypeptide that has substantially equivalent activity as compared to SEQ ID NO:2. By “modification” of the primary amino acid sequence it is meant to include “deletions” (that is, polypeptides in which one or more amino acid residues are absent), “additions” (that is, a polypeptide which has one or more additional amino acid residues as compared to the specified polypeptide), “substitutions” (that is, a polypeptide which results from the replacement of one or more amino acid residues), and “fragments” (that is, a polypeptide consisting of a primary amino acid sequence which is identical to a portion of the primary sequence of the specified polypeptide). By “modification” it is also meant to include polypeptides that are altered as a result of post-translational events which change, for example, the glycosylation, amidation, lipidation pattern, or the primary, secondary, or tertiary structure of the polypeptide.

It is known in the art that certain amino acids may be substituted by other amino acids having a similar hydropathic index or score and still result in a polypeptide having similar biological activity. In making such changes, the substitution of amino acids whose hydropathic indices are within ±2 is preferred, those that are within ±1 are more preferred, and those within ±0.5 are even more preferred. Similarly, select amino acids may be substituted by other amino acids having a similar hydrophilicity, as set forth in U.S. Pat. No. 4,554,101 (herein incorporated by reference in its entirety). In making such changes, as with the hydropathic indices, the substitution of amino acids whose hydrophilicity indices are within ±2 is preferred, those that are within ±1 are more preferred, and those within ±0.5 are even more preferred (See, e.g., Kyte et al., 157 J. Mol. Biol. 105-132 (1982), herein incorporated by reference in its entirety).

Conservative amino acid changes may be achieved by changing the codons of the DNA sequence using for example known redundancy in the code:

TABLE 1

| | Three-Letter | Single Letter | |
|---|---|---|---|
| Amino Acid | Designation | Designation | Codons |
| Alanine | Ala | A | GCA GCC GCG GCU |
| Cysteine | Cys | C | UGC UGU |
| Aspartic Acid | Asp | D | GAC GAU |
| Glutamic Acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In this aspect, the present invention also relates to vectors which comprise Pim-2, or variant thereof, and host cells which are genetically engineered with vectors of the invention, and to the production of Pim-2 polypeptides by recombinant techniques. Cell-free translation systems may also be employed to produce such proteins using RNAs derived from the DNA constructs of the present invention.

The Pim-2 polynucleotide of SEQ ID NO:1 may be obtained using standard cloning and screening, for example, from a cDNA library derived from mRNA or from genomic DNA libraries, or may be synthesized using well known and commercially available techniques. When the polynucleotide is used for recombinant production, that is to produce recombinant cells, it may consist of the mature polypeptide, or fragment thereof, of may include other coding sequences such as a leader or secretory sequence, a pre-, or pro- or pre-pro sequence, or other fusion peptide portions.

Host cells that are to be transformed may be genetically engineered to incorporate expression systems of the Pim-2 polypeptide. Introduction of the Pim-2 expression polynucleotide sequence into the host cell can be effectuated by any of the methods well known to those of ordinary skill in the art as described, for example, in Sambrook et al., Molecular Cloning: A Laboratory Manual, 2d Ed., Cold Spring Harbor Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection. Generally, any system or vector suitable to maintain, propagate or express the polynucleotide to produce the polypeptide may be used.

The present inventors have further demonstrated that the Pim-2 gene is a bona fide NF-κB target by virtue to its response to a transdominant IκBαSR (super repressor), and that its expression may be induced by lipopolysaccharide (“LPS”) (J. Biol. Chem. 276: 18579 (2001)). Studies performed by the present inventors suggest that up-regulation of Pim-2 in cells by LPS is controlled by the IKK/NF-κB pathway. The NF-κB signal transduction pathway involves a series of intracellular steps that promote phosphorylation and subsequent dissociation of IκB inhibitor protein from the inactive NF-κB complex. It is believed that liberated NF-κB translocates to the nucleus where it binds to the k enhancer element on the DNA and may activate transcription of Pim-2 gene. As the NF-κB signal transduction pathway is also induced by TNF, IL-1 and phorbol ester, these compounds as well may be used to induce Pim-2 activity.

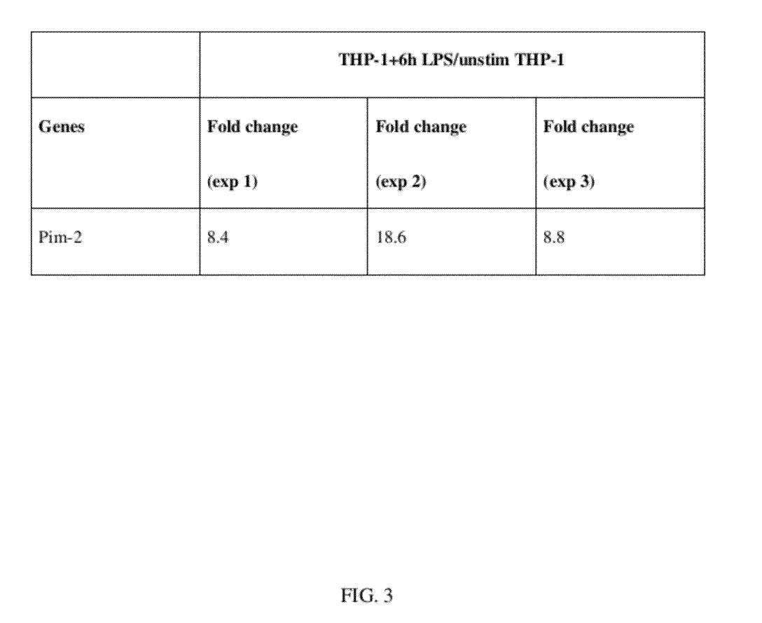

Expression with LPS of various cell lines, including human monocytes (THP-1) and mouse pre-B cells, has been seen by the present inventors to increase about 10 fold. An LPS (or other inducer of the NF-κB signal transduction pathway)-stimulated cell line may thus be used advantageously to improve the detection of compounds which may possess anti-inflammatory activity associated with decreased Pim-2 base line activity. FIG. 3 illustrates three different experiments undertaken to determine the fold change of Pim-2 RNA transcription in THP-1 cell lines, the fold change ranging from 8.4 to 18.6.

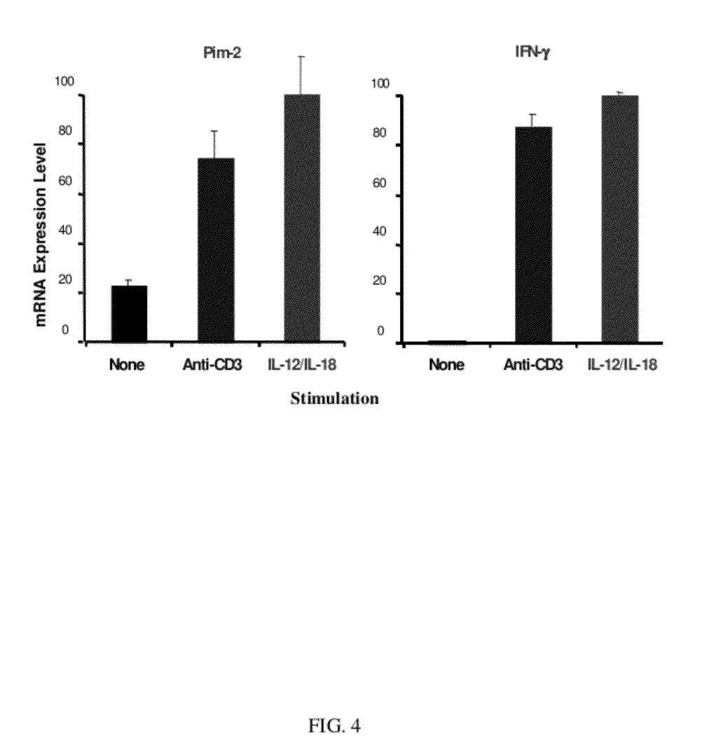

Crohn's Disease is mediated, inter alia, by activated Th1 cells. T cell cultures from patients with Crohn's Disease produce significantly higher levels of IFN-γ and TNF-α than T cell cultures from healthy controls (Agnholt, J. and K. Kaltoft, Cytokine 15(4):212-222 (2001)). FIG. 4 illustrates an experiment undertaken to determine induction of Pim-2 and IFN-γ by anti-CD3 or IL-12/IL-18 stimulation of CD4⁺ Th1 cells. As can be seen in FIG. 4, Pim-2 mRNA expression in stimulated CD4⁺ Th1 cells was significantly increased compared to unstimulated control cells.

Cell lines may alternatively, or may also, be activated to express Pim-2 by exposing the cells to Moloney murine leukemia proviruses.

Screening procedures may also entail a test to determine the binding of a candidate compound with Pim-2 itself. Binding may be detected by any of the methods well known in the art, as by means of a label directly or indirectly associated with a candidate compound or by measuring competition with a labeled competitor, such as an agonist of Pim-2 activity identified by the present invention which is found to bind to Pim-2 itself. Standard methods for conducting such screening assays are well understood in the art. Indicators of Pim-2 activity may include but are not limited to differences in kinase activity compared to control or changes in expression levels of pro-inflammatory substances, for example, TNF-α, IL-6, and IFN-γ.

### 4. Treatment of Inflammatory States Employing Agents Directed to Pim-2 Gene and Polypeptide

The polynucleotide sequence of Pim-2 is reported at GenBank Accession Nos. NM_{—}006875/U77735 based on sequence data reported by Baytel et al., Biochim. Biophys. Acta 1442: 274 (1998) (SEQ ID NO:3). The present inventors have discovered that the reported polynucleotide coding sequence of Pim-2 by Baytel et al. differs from that obtained by them in the sequencing of image EST clones comprising the Pim-2 gene. The Baytel et al. polynucleotide Pim-2 sequence indicates an additional thymidine (“t”) at position 1063 or 1064 in the coding region as opposed to SEQ ID NO:1, obtained by the present inventors, and a coding sequence at positions 185-1120, as opposed to a coding sequence 185 to 1117 obtained by the present inventors.

As is well known in the art, a single insertion (or deletion) in a nucleotide sequence compared to the actual sequence will cause a frame shift in translation of the nucleotide sequence such that the predicted amino acid sequence encoded by a determined nucleotide sequence will be different from the amino acid sequence actually encoded by the sequenced DNA molecule, beginning at the point of such an insertion (or deletion). The present inventors have determined that such is the case with respect to the amino acid sequence reported by Baytel et al. The Baytel et al. sequence (SEQ ID NO:4) indicates that Pim-2 has 41 amino acids not found in SEQ ID NO:2 and that it comprises twenty-three additional amino acids as compared to that SEQ ID NO:2 due to a belated stop codon. The present inventors have determined that SEQ ID NO:1 encodes wild-type Pim-2. The Baytel et al. polynucleotide sequence for Pim-2 may differ from wild-type due to the source material for its sequencing comprising a polymorphism in the form of an addition mutation, or may have resulted from a sequencing error.

Support for polypeptide SEQ ID NO:2 is found in genomics sequence data supplied by Ishida et al. at NCBI:AB042425 with respect to the genomic organization of human UDP-galactose transporter gene from which they predict a Pim-2 proto-oncogene “homolog” polypeptide (SEQ ID NO:5) that corresponds to the Pim-2 polypeptide uncovered by the present inventors. To date, there has been a lack of consistency in predicting human genes from genomic sequences (Hogenesch, et al., Cell 106: 413-415 (2001)). Because of potential errors of exons predicted from genomic sequences, Ishida et al. could not point out the mutation of sequence error in the cDNA record of h-Pim-2 (accession:U77735), instead, they named the predicted peptide sequence as a Pim-2 homologue. Support for polynucleotide SEQ ID NO:1, as well as polypeptide SEQ ID NO:2, is found at NCBI:XM_{—}010208 (SEQ ID NO:6) and NCBI:XP_{—}010208 (SEQ ID NO:7), both directly submitted by the National Center for Biotechnology, which based on sequence data derived by automated computational analysis of NCBI genomic sequence contig NT_{—}011611, using Assembly gene prediction methodology, indicate polynucleotide and polypeptide sequences corresponding to those found by the present inventors.

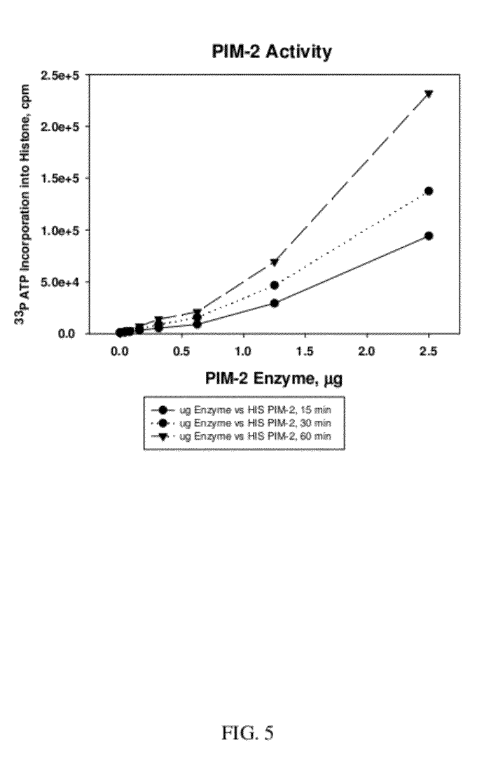

FIG. 5 illustrates that Pim-2 polypeptide as characterized by the present inventors is capable of phosphorylating histones in a concentration dependent manner.

The invention contemplates the amelioration and/or treatment of such diseases by administering a Pim-2 inhibiting amount of an inhibitor of Pim-2 activity. As would be understood by one of ordinary skill in the art, useful inhibitors of Pim-2 activity at the cellular level include, but are not limited to, compounds that inhibit the kinase activity of Pim-2, and/or reduce the expression of Pim-2 at either the transcription or translation level, and/or increase the degradation of Pim-2, and/or inhibit the interaction of Pim-2 with one or more of its upstream or downstream modulators/substrates, and include antibodies, or fragments or analogues thereof. In one embodiment of the present invention, the inhibitor of Pim-2 activity is identified by means of the screening test described above.

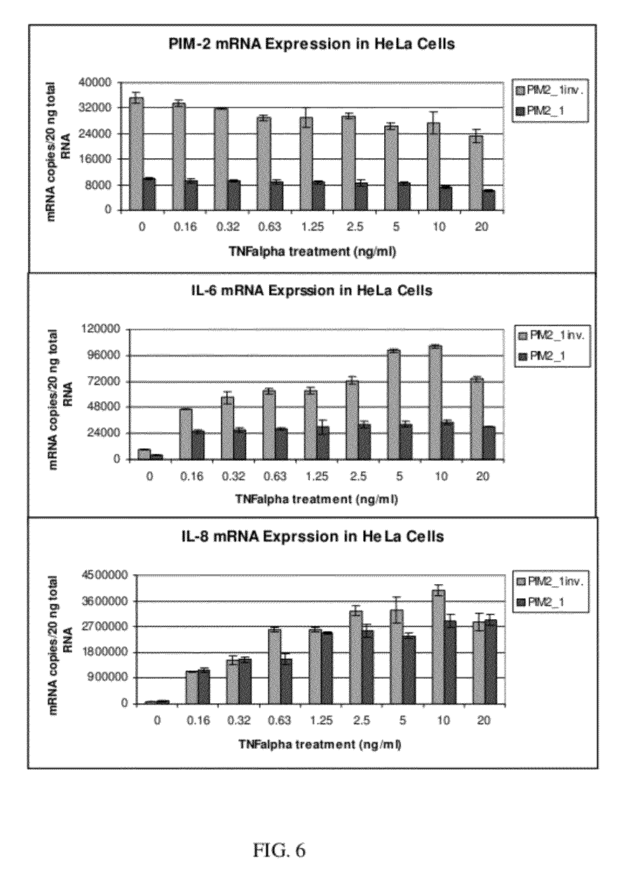

Inflammatory disease states may be treated by inhibiting the expression of the Pim-2 gene using expression blocking techniques, such techniques being known to those of ordinary skill in the art. For example, such techniques may involve the use of antisense sequences, either internally generated or separately administered (See, e.g., O'Connor, J. Neurochem. 56: 560 (1991) or the formation of triple helices with the gene (See, e.g., Dervan et al., Science 251:1360 (1991)). Alternatively, such techniques may utilize RNA interference (RNAi) technology (also referred to as short interfering RNA (siRNA) technology). FIG. 6 illustrates gene knock-down studies of Pim-2 in HeLa cells using the siRNA technology (Elbashir, S. M. et al, 2001, Nature, 411, 494-498). SiRNA oligo (Pim-2-1; sense: 5′-GUGAUUCCCCGGAAUCGUGTT-3′ (SEQ ID NO:8), antisense: 5′-CACGAUUCCGGGGAAUCACTT-3′ (SEQ ID NO:9) was designed to specifically knock down mRNA expression of Pim-2. The inverted siRNA oligo (Pim2-1 inv; sense: 5′-GUGCUAAGGCCCCUUAGUGTT-3′ (SEQ ID NO:10), antisense: 5′-CACUAAGGGGCCUUAGCACTT-3′ (SEQ ID NO:11)) was used as a control. The role of Pim-2 in mediating inflammation can at least be explained by its function in controlling the expression of interleukin-6 (IL-6). IL-6 is a major pro-inflammatory cytokine. Increased production of IL-6 has been reported in both Crohn's Disease and ulcerative colitis disease (Braegger C P et al., 1994, Ann Allergy, 72, 135-141). IL-6−/− mice display defective inflammatory response (Fattori E et al., J Exp Med 1994, 180:1243-1250). As shown in FIG. 6, when Pim-2 expression was suppressed by its siRNA oligo (Pim-2-1), the expression of IL-6 was reduced when cells were stimulated with various doses of TNF-α. Such repression is gene-specific, since the same siRNA had no significant effect on IL-8 production in response to TNF-α (FIG. 6).

Inflammatory disease states may also be treated by means of antibodies, or vaccines formulated to induce an immunological response in the affected animal so as to interfere with Pim-2 activity in the cell. For example, antibodies may be generated against the Pim-2 polypeptide of the present invention by administering Pim-2, or an epitope-bearing fragment thereof, to an animal capable of generating such antibodies using routine protocols. For preparation of monoclonal antibodies, any technique that provides antibodies produced by continuous cell line cultures can be used (See, e.g., Kohler and Milstein, Nature 256: 495 (1975)). Techniques for the production of single chain antibodies, as disclosed, for example, in U.S. Pat. No. 4,946,778, and be used to produce single chain antibodies to polypeptides of the invention. Non-human animals may further be used to express humanized antibodies. Vaccines may comprise inoculating a mammal with a Pim-2 polypeptide, or a fragment thereof, in adequate concentration to protect the animal from the inflammatory disease state, which is sought to be prevented. As polypeptides may be degraded in the gastric environment, proteinaceous vaccines are preferred to be administered parenterally.

Peptides or small molecules may be formulated in combination with a suitable pharmaceutical carrier. Carrier include, but are not limited to, saline, buffered saline, water, dextrose, glycerol, ethanol and combinations thereof. Formulation of the composition, of course, will depend upon the route of administration and the physical characteristics of the active. Formulation techniques are well known in the art. Any mode of administration may be employed so long as the active elicits an effect at the inflamed tissue, such administration including, without limitation, parenteral administration (including subcutaneous, intramuscular and intraperitoneal administration), enteral administration (including oral and rectal administration), dermal and transmucal administration, and ocular and aural administration. The invention further relates to pharmaceutical packs and kits comprising one or more containers filled with an active.

While the invention has been described with respect to certain embodiments, those skilled in the art will readily appreciate that various changes and/or modifications can be made to the invention without departing from the scope of the invention, and such changes and/or modifications are to be included within the spirit and purview of this application and the scope of the appended claims. All references cited in this specification are herein incorporated by reference to the same extent as if each individual reference was specifically and individually indicated to be incorporated by reference.

What is claimed is:

1. A method for detecting or diagnosing an inflammatory disease state using a tissue sample obtained from a patient, said method comprising the steps of:
(a) measuring a parameter indicative of the level of Pim-2 mRNA, in the tissue sample of the patient;
(b) determining any difference in the measurement of said parameter in the tissue sample of the patient as compared to the measurement of said parameter in comparable tissue sample(s) obtained from one or more patients lacking the inflammatory disease state.

2. The method of claim 1, further comprising the step of:
(c) diagnosing the patient as having said inflammatory disease state when said measurement of said parameter with respect to the patient's tissue is significantly higher than in said comparable tissue sample(s) obtained from said one or more patients lacking said inflammatory disease state.

3. The method of claim 1, wherein the method comprises in situ hybridization of at least one nucleic acid probe comprising a polynucleotide sequence of at least about 15 contiguous nucleotides of SEQ ID NO: 1.

4. The method of claim 3, wherein the polynucleotide sequence of said nucleic acid probe is derived includes nucleotides 294 through 311 of SEQ ID NO:1.

5. The method of claim 1, wherein the inflammatory disease state eventuates in an inflamed organ or segment thereof selected from the group consisting of: pancreas, tonsils, bowel, stomach lining, thyroid, cervix, lung, kidney, liver, and skin.


SEQUENCE LISTING (11 sequences)

SEQ ID NO: 1 - DNA, 1535 bp, Homo sapiens
  CDS (185)..(1117)
ggacgcgtgggcgcgcgcggcgaatctcaacgctgcgccgtctgcgggcgcttccgggccaccagtttctctgctttccaccctggcgccccccagccctggctccccagctgcgctgccccgggcgtccacgccctgcgggcttagcgggttcagtgggctcaatctgcgcagcgccacctccatgttgaccaagcctctacaggggcctcccgcgccccccgggacccccacgccgccgccaggaggcaaggatcgggaagcgttcgaggccgagtatcgactcggccccctcctgggtaaggggggctttggcaccgtcttcgcaggacaccgcctcacagatcgactccaggtggccatcaaagtgattccccggaatcgtgtgctgggctggtcccccttgtcagactcagtcacatgcccactcgaagtcgcactgctatggaaagtgggtgcaggtggtgggcaccctggcgtgatccgcctgcttgactggtttgagacacaggagggcttcatgctggtcctcgagcggcctttgcccgcccaggatctctttgactatatcacagagaagggcccactgggtgaaggcccaagccgctgcttctttggccaagtagtggcagccatccagcactgccattcccgtggagttgtccatcgtgacatcaaggatgagaacatcctgatagacctacgccgtggctgtgccaaactcattgattttggttctggtgccctgcttcatgatgaaccctacactgactttgatgggacaagggtgtacagccccccagagtggatctctcgacaccagtaccatgcactcccggccactgtctggtcactgggcatcctcctctatgacatggtgtgtggggacattccctttgagagggaccaggagattctggaagctgagctccacttcccagcccatgtctccccagactgctgtgccctaatccgccggtgcctggcccccaaaccttcttcccgaccctcactggaagagatcctgctggacccctggatgcaaacaccagccgaggatgtacccctcaacccctccaaaggaggccctgcccctttggcctggtccttgctaccctaagcctggcctggcctggcctggcccccaatggtcagaagagccatcccatggccatgtcacagggatagatggacatttgttgacttggttttacaggtcattaccagtcattaaagtccagtattactaaggtaagggattgaggatcaggggttagaagacataaaccaagtctgcccagttcccttcccaatcctacaaaggagccttcctcccagaacctgtggtccctgattctggagggggaacttcttgcttctcattttgctaaggaagtttattttggtgaagttgttcccattctgagccccgggactcttattctgatgatgtgtcaccccacattggcacctcctactaccaccacacaaacttagttcatatgctcttacttgggcaagggtgctttccttcc

SEQ ID NO: 2 - protein, 311 aa, Homo sapiens
MLTKPLQGPPAPPGTPTPPPGGKDREAFEAEYRLGPLLGKGGFGTVFAGHRLTDRLQVAIKVIPRNRVLGWSPLSDSVTCPLEVALLWKVGAGGGHPGVIRLLDWFETQEGFMLVLERPLPAQDLFDYITEKGPLGEGPSRCFFGQVVAAIQHCHSRGVVHRDIKDENILIDLRRGCAKLIDFGSGALLHDEPYTDFDGTRVYSPPEWISRHQYHALPATVWSLGILLYDMVCGDIPFERDQEILEAELHFPAHVSPDCCALIRRCLAPKPSSRPSLEEILLDPWMQTPAEDVPLNPSKGGPAPLAWSLLP

SEQ ID NO: 3 - DNA, 2088 bp, Homo sapiens
  CDS (186)..(1187)
  ref <301>: AUTHORS: Baytel, D. et al.
  ref <302>: TITLE: The human Pim-2 proto-oncogene and its testicular expression
  ref <303>: JOURNAL: Biochim. Biophys. Acta
  ref <304>: VOLUME: 2
  ref <305>: ISSUE: 1442
  ref <306>: PAGES: 274-285
  ref <307>: DATE: 1998
  ref <308>: DATABASE ACCESSION NUMBER: NM_006875
  ref <309>: DATABASE ENTRY DATE: 2000-11-02
  ref <313>: RELEVANT RESIDUES IN SEQ ID NO: RELEVANT RESIDUES: (1)..(2088)
gaattcggcacgagcgcgcggcgaatctcaacgctgcgccgtctgcgggcgcttccgggccaccagtttctctgctttccaccctggcgccccccagccctggctccccagctgcgctgccccgggcgtccacgccctgcgggcttagcgggttcagtgggctcaatctgcgcagcgccacctccatgttgaccaagcctctacaggggcctcccgcgccccccgggacccccacgccgccgccaggaggcaaggatcgggaagcgttcgaggccgagtatcgactcggccccctcctgggtaaggggggctttggcaccgtcttcgcaggacaccgcctcacagatcgactccaggtggccatcaaagtgattccccggaatcgtgtgctgggctggtcccccttgtcagactcagtcacatgcccactcgaagtcgcactgctatggaaagtgggtgcaggtggtgggcaccctggcgtgatccgcctgcttgactggtttgagacacaggaaggcttcatgctggtcctcgagcggcctttgcccgcccaggatctctttgactatatcacagagaagggcccactgggtgaaggcccaagccgctgcttctttggccaagtagtggcagccatccagcactgccattcccgtggagttgtccatcgtgacatcaaggatgagaacatcctgatagacctacgccgtggctgtgccaaactcattgattttggttctggtgccctgcttcatgatgaaccctacactgactttgatgggacaagggtgtacagccccccagagtggatctctcgacaccagtaccatgcactcccggccactgtctggtcactgggcatcctcctctatgacatggtgtgtggggacattccctttgagagggaccaggagattctggaagctgagctccacttcccagcccatgtctccccagactgctgtgccctaatccgccggtgcctggcccccaaaccttcttcccgaccctcactggaagagatcctgctggacccctggatgcaaacaccagccgaggatgttacccctcaacccctccaaaggaggccctgcccctttggcctggtccttgctaccctaagcctggcctggcctggcctggcccccaatggtcagaagagccatcccatggccatgtcacagggatagatggacatttgttgacttggttttacaggtcattaccagtcattaaagtccagtattactaaggtaagggattgaggatcaggggttagaagacataaaccaagtttgcccagttcccttcccaatcctacaaaggagccttcctcccagaacctgtggtccctgattttggagggggaacttcttgcttctcattttgctaaggaagtttattttggtgaagttgttcccattttgagccccgggactcttattttgatgatgtgtcaccccacattggcacctcctactaccaccacacaaacttagttcatatgcttttacttgggcaagggtgctttccttccaataccccagtagcttttattttagtaaagggaccctttcccctagcctagggtcccatattgggtcaagctgcttacctgcctcagcccaggattttttattttgggggaggtaatgccctgttgttaccccaaggcttctttttttttttttttttttttgggtgaggggaccctactttgttatcccaagtgctcttattctggtgagaagaaccttaattccataatttgggaaggaatggaagatggacaccaccggacaccaccagacaataggatgggatggatggttttttgggggatgggctaggggaaataaggcttgctgtttgttttcctggggcgctccctccaattttgcagatttttgcaacctcctcctgagccgggattgtccaattactaaaatgtaaataatcacgtattgtggggaggggagttccaagtgtgccctccttttttttcctgcctggattatttaaaaagccatgtgtggaaacccactatttaataaaagtaatagaatcagaaaaaaaaaaaaaaaaaaa

SEQ ID NO: 4 - protein, 334 aa, Homo sapiens
MLTKPLQGPPAPPGTPTPPPGGKDREAFEAEYRLGPLLGKGGFGTVFAGHRLTDRLQVAIKVIPRNRVLGWSPLSDSVTCPLEVALLWKVGAGGGHPGVIRLLDWFETQEGFMLVLERPLPAQDLFDYITEKGPLGEGPSRCFFGQVVAAIQHCHSRGVVHRDIKDENILIDLRRGCAKLIDFGSGALLHDEPYTDFDGTRVYSPPEWISRHQYHALPATVWSLGILLYDMVCGDIPFERDQEILEAELHFPAHVSPDCCALIRRCLAPKPSSRPSLEEILLDPWMQTPAEDVTPQPLQRRPCPFGLVLATLSLAWPGLAPNGQKSHPMAMSQG

SEQ ID NO: 5 - protein, 311 aa, Homo sapiens; identical to SEQ ID NO: 2
  PEPTIDE (1)..(311)
  ref <308>: DATABASE ACCESSION NUMBER: AB042425
  ref <309>: DATABASE ENTRY DATE: 2000-05-11
  ref <313>: RELEVANT RESIDUES IN SEQ ID NO: RELEVANT RESIDUES: (1)..(311)

SEQ ID NO: 6 - DNA, 2055 bp, Homo sapiens
  ref <308>: DATABASE ACCESSION NUMBER: XM_010208
  ref <309>: DATABASE ENTRY DATE: 2001-04-17
  ref <313>: RELEVANT RESIDUES IN SEQ ID NO: RELEVANT RESIDUES: (1)..(2055)
gcgcgcggcgaatctcaacgctgcgccgtctgcgggcgcttccgggccaccagtttctctgctttccaccctggcgccccccagccctggctccccagctgcgctgccccgggcgtccacgccctgcgggcttagcgggttcagtgggctcaatctgcgcagcgccacctccatgttgaccaagcctctacaggggcctcccgcgccccccgggacccccacgccgccgccaggaggcaaggatcgggaagcgttcgaggccgagtatcgactcggccccctcctgggtaaggggggctttggcaccgtcttcgcaggacaccgcctcacagatcgactccaggtggccatcaaagtgattccccggaatcgtgtgctgggctggtcccccttgtcagactcagtcacatgcccactcgaagtcgcactgctatggaaagtgggtgcaggtggtgggcaccctggcgtgatccgcctgcttgactggtttgagacacaggagggcttcatgctggtcctcgagcggcctttgcccgcccaggatctctttgactatatcacagagaagggcccactgggtgaaggcccaagccgctgcttctttggccaagtagtggcagccatccagcactgccattcccgtggagttgtccatcgtgacatcaaggatgagaacatcctgatagacctacgccgtggctgtgccaaactcattgattttggttctggtgccctgcttcatgatgaaccctacactgactttgatgggacaagggtgtacagccccccagagtggatctctcgacaccagtaccatgcactcccggccactgtctggtcactgggcatcctcctctatgacatggtgtgtggggacattccctttgagagggaccaggagattctggaagctgagctccacttcccagcccatgtctccccagactgctgtgccctaatccgccggtgcctggcccccaaaccttcttcccgaccctcactggaagagatcctgctggacccctggatgcaaacaccagccgaggatgtacccctcaacccctccaaaggaggccctgcccctttggcctggtccttgctaccctaagcctggcctggcctggcctggcccccaatggtcagaagagccatcccatggccatgtcacagggatagatggacatttgttgacttggttttacaggtcattaccagtcattaaagtccagtattactaaggtaagggattgaggatcaggggttagaagacataaaccaagtctgcccagttcccttcccaatcctacaaaggagccttcctcccagaacctgtggtccctgattctggagggggaacttcttgcttctcattttgctaaggaagtttattttggtgaagttgttcccattctgagccccgggactcttattctgatgatgtgtcaccccacattggcacctcctactaccaccacacaaacttagttcatatgctcttacttgggcaagggtgctttccttccaataccccagtagcttttattttagtaaagggaccctttcccctagcctagggtcccatattgggtcaagctgcttacctgcctcagcccaggattctttattctgggggaggtaatgccctgttgttaccccaaggcttcttttttttttttttttttttgggtgaggggaccctactctgttatcccaagtgctcttattctggtgagaagaaccttacttccataatttgggaaggaatggaagatggacaccaccggacaccaccagacactaggatgggatggatggttttttgggggatgggctaggggaaataaggcttgctgtttgttctcctggggcgctccctccaacttttgcagattcttgcaacctcctcctgagccgggattgtccaattactaaaatgtaaataatcacgtattgtggggaggggagttccaagtgtgccctcctctcttctcctgcctggattatttaaaaagccatgtgtggaaacccactatttaataaaagtaatagaatcag

SEQ ID NO: 7 - protein, 311 aa, Homo sapiens; identical to SEQ ID NO: 2
  ref <308>: DATABASE ACCESSION NUMBER: XP_010208
  ref <309>: DATABASE ENTRY DATE: 2001-07-12
  ref <313>: RELEVANT RESIDUES IN SEQ ID NO: RELEVANT RESIDUES: (1)..(311)

SEQ ID NO: 8 - DNA, 21 bp, Homo sapiens
  (1)..(21): Short interfering RNA construct comprising combined DNA/RNA
  ref <313>: RELEVANT RESIDUES IN SEQ ID NO: RELEVANT RESIDUES: (1)..(21)
gugauuccccggaaucguctt

SEQ ID NO: 9 - DNA, 21 bp, Homo sapiens
  (1)..(21): Short interfering RNA construct comprising combined DNA/RNA
  ref <313>: RELEVANT RESIDUES IN SEQ ID NO: RELEVANT RESIDUES: (1)..(21)
cacgauuccggggaaucactt

SEQ ID NO: 10 - DNA, 21 bp, Homo sapiens
  (1)..(21): Short interfering RNA construct comprising combined DNA/RNA
  ref <313>: RELEVANT RESIDUES IN SEQ ID NO: RELEVANT RESIDUES: (1)..(21)
gugcuaaggccccuuagugtt

SEQ ID NO: 11 - DNA, 21 bp, Homo sapiens
  (1)..(21): Short interfering RNA construct comprising combined DNA/RNA
  ref <313>: RELEVANT RESIDUES IN SEQ ID NO: RELEVANT RESIDUES: (1)..(21)
cacuaaggggccuuagcactt